(19) United States
(12) Patent Application Publication
(10) Pub. No.: US 2023/0030449 A1
(43) Pub. Date: Feb. 2, 2023

(54) STENT

(71) Applicant: SB-KAWASUMI LABORATORIES, INC., Kanagawa (JP)

(72) Inventors: Ryota TSUKAMOTO, Kanagawa (JP); Seiichi NAKAYA, Kanagawa (JP)

(21) Appl. No.: 17/786,638

(22) PCT Filed: Dec. 14, 2020
(86) PCT No.: PCT/JP2020/046556

§ 371 Date: Jun. 17, 2022

(30) Foreign Application Priority Data
Dec. 25, 2019 (JP) 2019-234361

(51) Int. Cl. A61F 2/856; A61F 2/954

(52) U.S. Cl. CPC A61F 2/856; A61F 2/954; A61F 2250/006; A61F 2250/0039

(57) ABSTRACT

Provided is a stent that can be easily and reliably placed in a branched portion of a biological lumen. A stent is configured to be placed within a biological lumen and comprises: a first skeleton which is for placement within a first lumen of the biological lumen, and which is made of a wire material and formed into a tubular shape; and second skeletons which are for placement within a plurality of second lumens branched from the first lumen, and which are made of a wire material different from that of the first skeleton and whish are formed into a tubular shape. The plurality of second skeletons are provided so as to branch from one end section of the first skeleton and are engaged with each other at the branched portion.

### TECHNICAL FIELD

The present invention relates to a stent.

### BACKGROUND ART

Conventionally, several stents have been known which is placed in a stenotic part or an occlusive part occurring in a biological lumen such as the blood vessel, esophagus, bile duct, trachea, or ureter, and increases in diameter of the lesion site to maintain an opening state of the biological lumen. In a stent graft placement technique, a stent is sometimes branched to be placed depending on condition of the lesion site. For example, a lesion occurring in the vicinity of the hepatoportal needs a placement of a stent at each of the common hepatic duct, the right hepatic duct and the left hepatic duct, since the common hepatic duct branches into the right hepatic duct and the left hepatic duct (intrahepatic bile ducts).

In such a case, a plurality of stents has been conventionally prepared so as to include a stent for the main lumen (e.g., for the common hepatic duct) and stents for branched lumens (e.g., for the right hepatic duct and left hepatic duct), and then one of the stents receives, in its opening (e.g., a mesh of a skeleton portion), insertion of another stent to connect together the stents with partially overlapping each other (see e.g., Patent Document 1). For example, in a placement of a stent for a lesion site occurring in the vicinity of the hepatoportal, a stent placed extending from the common hepatic duct to one hepatic duct (e.g., the right hepatic duct) receives insertion of and is connected to a stent to be placed in the other hepatic duct (e.g., the left hepatic duct).

### Prior Art Document

### Patent Document

Patent Document 1: Japanese Unexamined Patent Application Publication: 2014-138851

### SUMMARY OF THE INVENTION

### Technical Problem

However, in the context of Patent Document 1, each stent requires a placement system, and furthermore, techniques in a placement of a stent are complicated, thus possibly leading to deformation or breakage of a stent and occlusion in the hepatic portal part. In addition, a tangle is generated among meshes of a stent, and makes post-placement evulsion difficult. Therefore, an operator that performs a stent placement technique needs to have extensive experience and high skills.

An object of the present invention is to provide a stent that is capable of being placed in a branch part of a biological lumen easily and reliably.

### Solution to Problem

A stent according to the present invention is: a stent placed inside a biological lumen including: a first skeleton to be placed inside a first lumen in the biological lumen, the first skeleton being formed of a wire into a hollow shape, and a plurality of second skeletons to be placed in a plurality of second lumens branching from the first lumen, the plurality of second skeletons each being formed of a wire different from that of the first skeleton into hollow shapes, wherein the plurality of second skeletons is disposed so as to branch from one end of the first skeleton and engages with one another at a branch part.

### Advantageous Effect of the Invention

The present invention allows an easy, reliable placement of a stent in a branch part.

### BRIEF DESCRIPTION OF THE DRAWINGS

FIG. 1 shows an appearance of a bile duct stent according to the embodiment.

FIG. 2A and FIG. 2B show an example of a placement aspect of the bile duct stent.

FIG. 3 schematically shows a cross section including engaging parts of a first skeleton with second skeletons and an engaging part of the second skeletons.

FIG. 4A and FIG. 4B schematically show a placed state of the bile duct stent in the hepatic portal part.

FIG. 5A and FIG. 5B show a modified example of the bile duct stent.

### DESCRIPTION OF THE EMBODIMENT

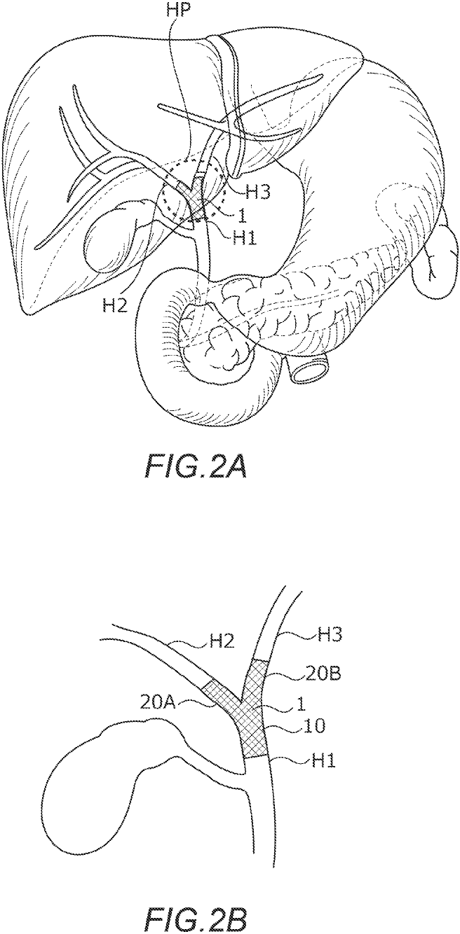

Hereinafter, embodiments of the present invention will be described in detail with reference to the drawings. In the embodiments, description will be made for a bile duct stent 1, as an example of the present invention, which is placed and used inside a common hepatic duct H1, a right hepatic duct H2, and a left hepatic duct H3 in order to push and expand radially outward a lesion site in a hepatic portal part HP (see FIG. 2A and FIG. 2B) (e.g., an occlusive part or stenotic part in the hepatic portal part HP) to treat occlusion (stenosis).

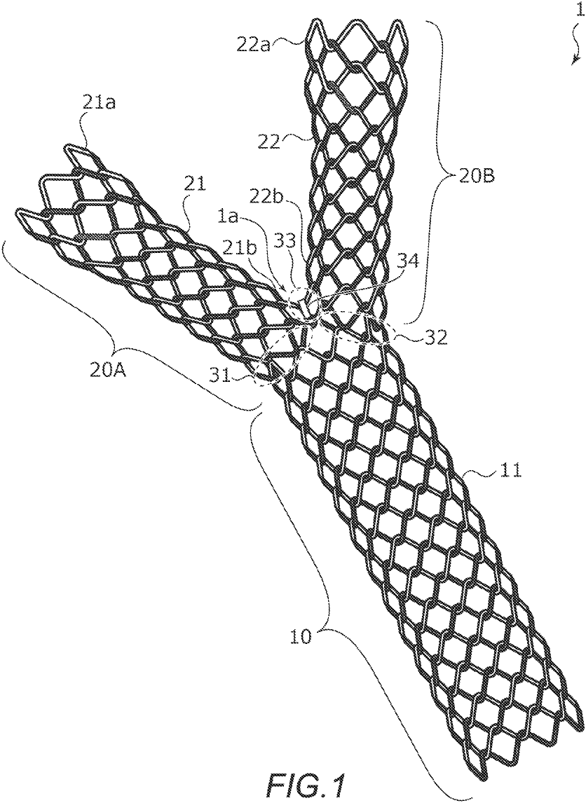

FIG. 1 shows an appearance of the bile duct stent 1 according to the embodiment. FIG. 2A and FIG. 2B show a placement state of the bile duct stent 1. FIG. 2B shows the hepatic portal part HP in FIG. 2A as an enlarged view.

The bile duct stent 1 is so-called a bare stent sorely consisting of a skeleton. The bile duct stent 1 is divided into a first stent portion 10 and second stent portions 20A and 20B. As shown in FIG. 2A and FIG. 2B, the first stent portion 10 and the second stent portion 20A and 20B are placed in the common hepatic duct H1, the right hepatic duct H2, and the left hepatic duct H3, respectively, and push and expand the lumens to define a bile flow path.

The second stent portions 20A and 20B are connected to one end of the first stent portion 10 so as to form bifurcation. In other words, the bile duct stent 1 has a Y-shape as a whole. The angle of a stent branching part 1a as a fork part branching into the second stent portions 20A and 20B is set corresponding to a shape of the hepatic portal part HP where the bile duct stent 1 is to be placed.

Although illustration is omitted, the bile duct stent 1 may have an evulsion assistant portion to catch an evulsion wire, at the end on a side to be drawn into a retract tube at evulsion.

The first stent portion 10 has a first skeleton 11 formed of a wire into a hollow shape. The second stent portions 20A and 20B have second skeletons 21 and 22 formed of a wire different from that of the first skeleton 11 into hollow shapes. In addition, the second skeletons 21 and 22 may be formed of the same wire or formed of different wires. The tube diameters of the first skeleton 11 and the second skeletons 21 and 22 are set corresponding to the tube diameters of the common hepatic duct H1, the right hepatic duct H2, and the left hepatic duct H3, respectively, to receive placements. For example, the tube diameter of the second skeletons 21 and 22 may be set to be smaller than the tube diameter of the first skeleton 11.

The first skeleton 11 and the second skeletons 21 and 22 are, for example, formed by braiding a wire into a tubular shape so as to control axial elongation. In detail, the first skeleton 11 and the second skeletons 21 and 22 are formed by braiding into a rhombic metal-net shape (fence shape) two wires spirally extending with folding in a zigzag (Z-shaped) pattern at a predetermined pitch, so as to make bends (a mountain projected to one axial end on one side and a valley projected to the other axial end on the other side) engage with each other. While tension is axially applied to the bile duct stent 1, bends of a wire forming a mesh cross densely one another, thus controlling axial elongation.

The first skeleton 11 and the second skeletons 21 and 22 have so-called self-expandability, which is to memorize a shape in an expanded state, and expands radially outward along with release from a sheath (depiction omitted). In other words, the first stent portion 10 and the second stent portions 20A and 20B are configured to be deformable from a contracted state where they are folded radially inward, to an expanded state where they expand radially outward to define a tubular flow path.

Examples of materials of the wires forming the first skeleton 11 and the second skeletons 21 and 22 include known metals or metal alloys typified by stainless steel, Ni-Ti alloy (nitinol), titanium alloy, and the like. Alloy materials having X-ray contrast property may also be used. In this case, the position of the bile duct stent 1 can be determined from outside the body. Here, the first skeleton 11 and the second skeletons 21 and 22 may be formed of a material other than metal materials (e.g., ceramic or resin).

In addition, materials of wires forming the first skeleton 11 and the second skeletons 21 and 22, the diameter of the wire (cross-sectional area), the number of folds and the shape of folds (the number and the shape of bends) in the circumferential direction, the mesh size (the skeleton amount per unit length), and the like are appropriately selected on the basis of expandability and flexibility of the first stent portion 10 and the second stent portions 20A and 20B to be required corresponding to a biological lumen to receive placements. In this regard, flexibility refers to ease of bending of the first stent portion 10 and the second stent portions 20A and 20B, and is particularly defined by flexural rigidity in the axial direction. In other words, high flexibility of the first stent portion 10 and the second stent portions 20A and 20B refers to having appropriately low flexural rigidity in the axial direction, and a characteristic of following a shape of a biological lumen or a sheath without kinking inside the biological lumen or the sheath.

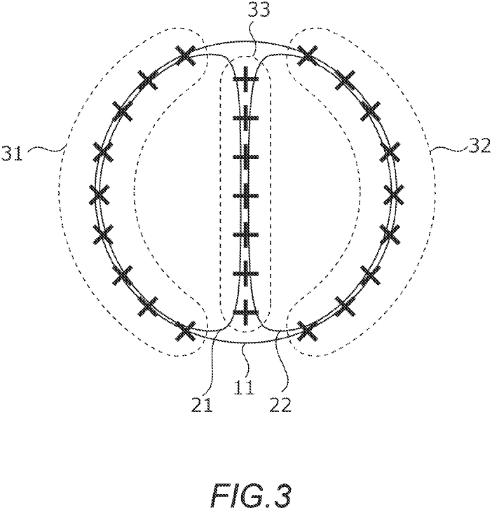

Moreover, the second skeletons 21 and 22 are engaged and combined with the first skeleton 11 at fixed ends 21b and 22b (joint parts) in the vicinity of the first stent portion 10. The second skeletons 21 and 22 are engaged with the first skeleton 11 at the semicircles of the fixed ends 21b and 22b, respectively (see FIG. 3). FIG. 3 schematically shows a cross section including engaging parts 31 and 32 of the first skeleton 11 with the second skeleton 21 and 22 and an engaging part 33 of the second skeletons 21 and 22.

For example, the second skeletons 21 and 22 are engaged with the first skeleton 11 by beginning to braid the second skeletons 21 and 22 with passing wires of the second skeletons 21 and 22 through meshes formed at the end of the first skeleton 11. In this regard, after the second skeletons 21 and 22 are formed separately from the first skeleton 11, a caulking member (depiction omitted) may be used to connect the second skeletons 21 and 22 to the first skeleton 11.

Furthermore, the fixed ends 21b and 22b of the second skeletons 21 and 22 are engaged and combined, and is incapable of being separated (see FIG. 3). For example, mutual contact parts of the fixed ends 21b and 22b of the second skeletons 21 and 22 are joined by a caulking member 34, thereby engaging the fixed ends 21b and 22b of the second skeletons 21 and 22. In the contact parts of the fixed ends 21b and 22b of the second skeletons 21 and 22, meshes of both parts may cross each other. In this case, the second skeletons 21 and 22 are only joined loosely at their joints, thus has a high degree of freedom, is capable of being loaded easily in a sheath, and readily adapts to a shape of a bile duct branching part (a branching angle) that branches from the common hepatic duct H1 into the right hepatic duct H2 and the left hepatic duct H3.

Thus, in the bile duct stent 1, the first skeleton 11 and the second skeletons 21 and 22 are interlinked without radially overlapping. In other words, the bile duct stent 1 has a structure different from a conventional partial stent-in-stent form with a plurality of stents connected to make the stents overlap partially each other. Since the bile duct stent 1 is formed so as to be capable of being placed inside the common hepatic duct H1, the right hepatic duct H2, and the left hepatic duct H3 with integrating the first stent portion 10 and the second stent portions 20A and 20B, the bile duct stent 1 can be placed easily in the hepatic portal part HP (a branch part of the biological lumen) with a single manipulation. Accordingly, a stable operation can be achieved regardless of experiences or skills of an operator.

Moreover, the second skeletons 21 and 22 have larger tube diameters at free ends 21a and 22a than tube diameters at fixed ends 21b and 22b on opposite sides in the axial directions. In other words, the free end 21a and 22a of the second skeletons 21 and 22 exhibit so-called flare shapes and have larger expansion force than the fixed ends 21b and 22b. In a placement of the bile duct stent 1, the second stent portions 20A and 20B are released from a sheath, followed by releasing the first stent portion 10. That means, the free ends 21a and 22a of the second skeletons 21 and 22 are released at first. Forming the free ends 21a and 22a of the second skeletons 21 and 22 into flare shapes improves adhesion to the right hepatic duct H2 and the left hepatic duct H3, thus enabling prevention of generation of a position gap at a placement of the bile duct stent 1, and allowing the bile duct stent 1 to be placed at a proper position.

In addition, the caulking member 34 for engaging the second skeletons 21 and 22 preferably functions as a marker to be an index inside the biological lumen. For example, the caulking member 34 can be formed of an alloy material having X-ray contrast property and thereby function as a marker. This enables, with use of the caulking member 34 for engagement, checking whether the engaging part 33 of the second skeletons 21 and 22, i.e., the stent branching part 1a of the bile duct stent 1, is properly located at a bile duct branching part. This case does not require presence of a marker for checking a position of the bile duct stent 1 inside the biological lumen, thus simplifying the bile duct stent 1 and improving containability into and releasability from a sheath.

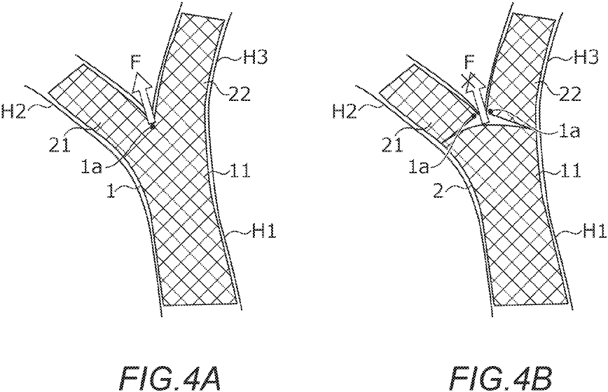

FIG. 4A and FIG. 4B schematically show placement states of the bile duct stents 1 and 2 in the hepatic portal part HP. FIG. 4A depicts a placement state of the bile duct stent 1 according to the embodiment, and FIG. 4B depicts a placement state of the bile duct stent 2 without engagement with the second skeletons 21 and 22.

In the bile duct stent 2, the second skeletons 21 and 22 are engaged only with the first skeleton 11, but the second skeletons 21 and 22 are not engaged with each other. Accordingly, when the bile duct stent 2 is released from a sheath, joints of the second skeletons 21 and 22 are separated, thus likely to fail to properly place the bile duct stent 2 throughout the hepatic portal part HP (see FIG. 4B). Furthermore, in evulsion of the bile duct stent 2 placed, an unengaging part of the joint of the second skeletons 21 and 22 is likely to be caught on the lumen wall to prevent an evulsion operation. Even if the bile duct stent 2 can be placed with the joints of the second skeletons 21 and 22 contacting closely, expansion force F is not to act on a bile duct branching part. The bile duct stent 2 is thus incapable of treating occlusion (stenosis) of a bile duct branching part.

By contrast, the bile duct stent 1 is engaged with the second skeletons 21 and 22, and made to restrict their individual position (shape) even when the bile duct stent 1 is released from a sheath. This allows easy positioning of the stent branching part 1a in a bile duct branching part (see FIG. 4A). Thus the bile duct stent 1 can be placed properly at a desired placement site, making the joint parts of the second skeletons 21 and 22 less likely to be caught on the lumen wall in evulsion. Moreover, the expansion force F also acts on a bile duct branching part, thus allowing treatment not only of occlusion (stenosis) generated in the common hepatic duct H1, the right hepatic duct H2, or the left hepatic duct H3, but also of occlusion (stenosis) generated in a bile duct branching part.

As described above, the bile duct stent 1 according to the embodiment is a stent to be placed inside the hepatic portal part HP (biological lumen), and includes the first skeleton 11, which is to be placed inside the common hepatic duct H1 (first lumen) and is tubularly formed of a wire, and the second skeletons 21 and 22, which are to be placed inside the right hepatic duct H2 and the left hepatic duct H3 (a plurality of second lumens) branching from the common hepatic duct H1 and is tubularly formed of a wire different from that of the first skeleton 11. The second skeletons 21 and 22 are disposed so as to branch from one end of the first skeleton 11 and engaged with each other in the stent branching part 1a (branch part).

The bile duct stent 1 allows easy, reliable positioning of the stent branching part 1a in a bile duct branching part, and proper placement of the bile duct stent 1 at a desired placement site, as well as attempt to facilitate evulsion operation after a placement. Moreover, the expansion force F also acts on a bile duct branching part, thus allowing treatment not only of occlusion (stenosis) generated in the common hepatic duct H1, the right hepatic duct H2, or the left hepatic duct H3, but also of occlusion (stenosis) generated in a bile duct branching part.

In the bile duct stent 1, the second skeletons 21 and 22 have larger tube diameters at the free ends 21a and 22a on sides opposite to the first skeleton 11 in the axial direction than tube diameters at the fixed ends 21b and 22b on the first skeleton 11 side. This provides the free ends 21a and 22a of the second skeletons 21 and 22 with expansion force larger than expansion force of the fixed ends 21b and 22b, thereby enabling prevention of generation of a position gap in a placement, and allowing the bile duct stent 1 to be placed properly at a desired placement site.

Additionally, in the bile duct stent 1, the second skeletons 21 and 22 are braided so as to control axial elongation. This secures expansion force in the stent branching part 1a, thus allowing a bile duct branching part to be appropriately pushed and expanded. Furthermore, the axial length of the bile duct stent 1 does not significantly change in a placement, thus allowing the bile duct stent 1 to be placed properly in a desired placement site.

Moreover, in the bile duct stent 1, the second skeletons 21 and 22 are engaged by the caulking member 34 (engaging member), and the caulking member 34 functions as a marker to be an index inside the biological lumen. This enables, with use of the caulking member 34 for engagement, checking whether the stent branching part 1a of the bile duct stent 1 is properly located at a bile duct branching part.

Furthermore, in the bile duct stent 1, the first skeleton 11 and the second skeletons 21 and 22 are engaged. This provides presence of an additional skeleton in a border of the first stent portion 10 and the second stent portions 20A and 20B and secures expansion force in this part, thus allowing the bile duct stent 1 to be placed properly.

The invention made by the inventors has been particularly described on the basis of the embodiments so far, but the present invention is not limited to the embodiments described above, and can be changed within the scope not departing from the spirit.

For example, in the bile duct stent 1, the first skeleton 11 and the second skeletons 21 and 22 may not include a rhombic metal-net shape, but may have a configuration where one or more wires are braided by bending so as to form alternately mountain and valleys, as well as winding spirally in each axial direction.

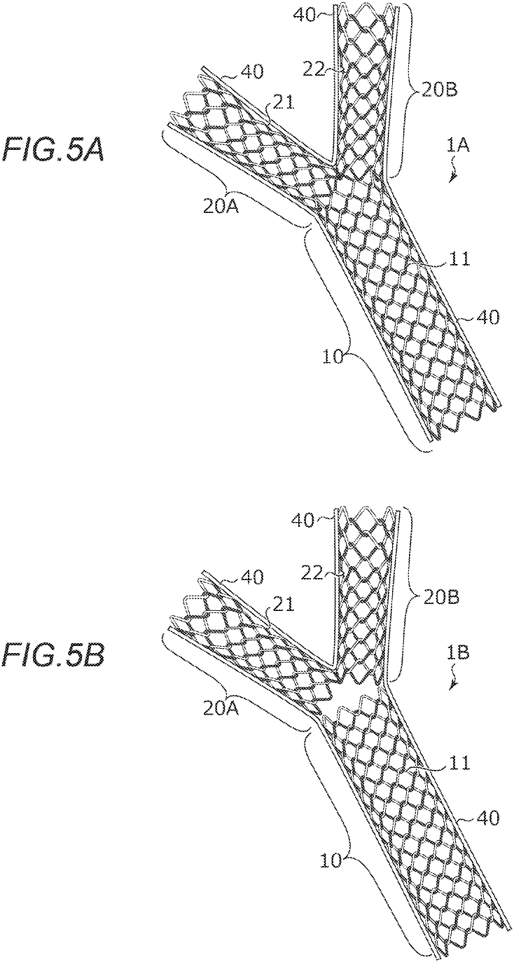

For another example, the first stent portion 10 and the second stent portions 20A and 20B have a film 40 covering the first skeleton 11 and the second skeletons 21 and 22, respectively, and the film 40 may integrate the first stent portion 10 and the second stent portions 20A and 20B, as a bile duct stent 1A shown in FIG. 5A. Examples of materials forming the film 40 include silicone resin, fluorine resin such as PTFE (polytetrafluoroethylene), and polyester resin such as polyethylene terephthalate. When the film 40 is provided, the first skeleton 11 may not be engaged with the second skeletons 21 and 22, as a bile duct stent 1B shown in FIG. 5B. The first stent portion 10 and the second stent portions 20A and 20B can be integrated regardless of forms of the first skeleton 11 and the second skeletons 21 and 22, thus improving the degree of freedom for design.

Note that the configuration of the film 40 can be changed appropriately and freely. For example, the films 40 may be disposed on the outer peripheral face and the inner peripheral face of the skeletons so as to interpose the first skeleton 11 and the second skeletons 21 and 22, or may be only disposed on the outer peripheral face of the skeletons, or may be only disposed on the inner peripheral face. For another example, the film 40 may be disposed in any of the first stent portion 10 and the second stent portions 20A and 20B, or the film 40 may be disposed wholly or partially in each of them.

Moreover, when the film 40 is disposed in the first stent portion 10, the first skeleton 11 may not take a braided form that stands by itself by a skeleton only and exhibits a tubular shape, and for example, can also have a configuration where a plurality of skeletons formed annularly with bending wires so as to form alternately mountains and valleys is fixed to the film 40 at a predetermines interval in axial direction.

Additionally, the bile duct stent 1 is set to have two second skeletons 21 and 22, but is by way of example and not limited thereto. In other words, the number of the second skeletons can be appropriately changed freely, and three or more second skeletons may be included. Furthermore, in a branch part, all of three or more second skeletons may not engage with each other, and at least two second skeletons only have to engage with each other.

The present invention is not limited to the bile duct stent 1 described in the embodiments, but can be applied to a stent to be placed in a branch part of a biological lumen such as a gastrointestinal system lumen or blood vessel.

It should be understood that the embodiments disclosed herein are illustrative in all respects and are not restrictive. The scope of the present invention is indicated not by the above description but by the claims, and it is intended to encompass all modifications within the spirit and scope equivalent to the claims.

The content of disclosure of the specification, drawings, and abstract in Japanese Patent Application No. 2019-234361 filed on Dec. 25, 2019 is incorporated herein in their entirety.

### DESCRIPTION OF REFERENCE NUMERALS

1 bile duct stent (stent)
1a stent branching part 10 first stent portion
11 first skeleton
20A, 20B second stent portion
21, 22 second skeleton
31, 32, 33 engaging part
40 film
HP hepatic portal part (biological lumen)
H1 common hepatic duct (first lumen)
H2 right hepatic duct (second lumen)
H3 left hepatic duct (second lumen)

1. A stent placed inside a biological lumen, the stent comprising:
a first skeleton to be placed inside a first lumen in the biological lumen, the first skeleton being formed of a wire into a hollow shape, and
a plurality of second skeletons to be placed in a plurality of second lumens branching from the first lumen, the plurality of second skeletons each being formed of a wire different from that of the first skeleton into hollow shapes,
wherein the plurality of second skeletons is disposed so as to branch from one end of the first skeleton and engages with one another at a branch part.

2. The stent according to claim 1, wherein at least one of the plurality of second skeletons has a larger tube diameter at a free end on a side opposite to a fixed end on the first skeleton side in an axial direction than a tube diameter at the fixed end.

3. The stent according to claim 1, wherein the plurality of second skeletons is formed by braiding so as to control axial elongation.

4. The stent according to claim 1,
wherein the plurality of second skeletons are engaged by an engaging member, and
wherein the engaging member functions as a marker to be an index inside the biological lumen.

5. The stent according to claim 1, wherein the first skeleton and the plurality of second skeletons are engaged with each other.
